(19) United States
(12) Patent Application Publication
(10) Pub. No.: US 2014/0199679 A1
(43) Pub. Date: Jul. 17, 2014

(54) BIOREACTOR

(71) Applicants: Nicki Panoskaltsis, Middlesex (GB); Hugo Miguel Magalhäes Macedo, Albufeira (PT); Maria Teresa Mortera Blanco, Greater London (GB); Athanasios Mantalaris, South Kensington (GB); Andrew Guy Livingston, Welwyn Garden City (GB)

(72) Inventors: Nicki Panoskaltsis, Middlesex (GB); Hugo Miguel Magalhäes Macedo, Albufeira (PT); Maria Teresa Mortera Blanco, Greater London (GB); Athanasios Mantalaris, South Kensington (GB); Andrew Guy Livingston, Welwyn Garden City (GB)

(21) Appl. No.: 13/989,165

(22) PCT Filed: Nov. 24, 2011
(86) PCT No.: PCT/GB2011/052316

§ 371 Date: Feb. 27, 2014

(30) Foreign Application Priority Data
Nov. 24, 2010 (GB) 1019932.1

(51) Int. Cl. C12N 5/078; C12M 1/00

(52) U.S. Cl. CPC C12N 5/0641; C12M 47/04
USPC 435/2; 435/297.1; 435/297.4

(57) ABSTRACT

A bioreactor for the formation of mature blood cells from haematopoietic stem cells is disclosed. The bioreactor comprises a first zone and a second zone. The first zone and the second zone are separated by a first membrane. The first membrane allows the preferential passage of red blood cells relative to the haematopoietic stem cells and their 5 other progeny excluding red blood cells. The first membrane is formed by at least a separating layer and a porous layer, where the porous layer is in contact with the first zone, such that the haematopoietic stem cells can be grown in the porous layer. The bioreactor comprises a third zone. The first zone and the third zone are separated by a second membrane, and the second membrane allows the passage of nutrients from the 10 third zone to the first zone and the passage of metabolites of the cells from the first zone to the third zone, while substantially preventing the passage of growth factors from the first zone to the third zone.

This invention relates to a bioreactor, to the use of the bioreactor and to processes using the bioreactor.

### BACKGROUND

This invention relates to the field of tissue engineering, and more specifically to the field of blood production. The successful transfer of stem cell technology and cellular products into widespread clinical applications needs to address issues of cost, automation, standardisation and generation of clinically-relevant cell numbers of high quality. Laboratories and industry alike have dealt with similar problems in the past through the use of bioreactors. Consequently, stem cell bioprocessing will involve the use of specialized bioreactor devices that need to facilitate mass transport, high cell density, monitoring and feedback, and tissue-specific functional specialisation, thus mimicking the ultimate bioreactors which are the tissues/organs within the human body. A successful culture environment would provide proper conditions for the proliferation and maintenance of viability of the cells of interest; depending on the final intention; it should also be able to provide conditions that can promote targeted commitment and/or differentiation in cases where these cells have the capacity to undertake such pathways.

A particular case regards the expansion of stem cells, usually found in relatively low amounts in the adult body (adult stem cells) or requiring high numbers of embryo sacrifices. However, large numbers are usually required for clinical applications. This highlights the need for the in vitro expansion of stem cells prior to their commitment into tissue-specific applications. A very interesting example of this can be seen in the the human bone marrow (BM), whose engineering mimicry could open virtually unlimited doors of opportunities in the field of medicine and life quality.

The BM is a highly functional and complex organ within which a myriad of important biological processes take place in parallel. It is so sensitive and fundamental for the individual's life that it resides within the protection of the bone shell. Within this vital organ, resident stem cells, committed to the blood cells' lineage (the so-called hematopoietic stem cells or HSCs), have the task of replenishing the numerous blood cells that every day enter apoptosis in the body (on average, the body of a man weighing 70 Kg produces around 10¹² blood cells per day). These progenitor cells, as for every stem cell, retain two characteristic functions: the ability to self-renew (thus the capacity to expand into greater numbers of cells) and to undertake differentiation (arising into the specialized and non-proliferative mature blood cells). These are two processes which require a very specialized environment, where both physical and biochemical signals are important for their regulation as well as viability.

A close and intricate net of different molecules and accessory cells, such as stromal cells, the extracellular matrix (ECM) proteins and an array of soluble and ECM-bound growth factors, also known as the Hematopoietic Inductive Microenvironment (HIM), promote the necessary biochemical support. On the other hand, an intricate three-dimensional (3D) architecture provides the physical support, allowing a higher surface for cellular adhesion while promoting closer contact between the cells, not only to exchange biochemical signals between each other but also with the surrounding microenvironment. The ECM is the skeleton of the in vivo niche that protects and supplies all the cells residing within it, forming a specialized microenvironment. It is secreted by virtually every cell in the body, and is responsible for several general tasks: mechanical support for cell anchorage; determination of cell orientation; control of cell growth; maintenance of cell differentiation; scaffolding for orderly tissue renewal; establishment of tissue microenvironment; and sequestration, storage and presentation of soluble regulatory molecules.

However, this 3D architecture has the downside of offering high resistance to the mass transfer required to renew this microenvironment (supply of nutrients, oxygen and other important molecules depleted during the cellular metabolism and removal of the metabolites, carbon dioxide and other debris). To have an idea of this, a cylinder with 1 cm diameter composed of material similar to a frog's nerve, if suddenly placed in oxygen, would take 185 minutes to attain 90 per cent of its full saturation with that gas. However, the vascularization of this structure in vivo is responsible for promoting a faster intake, and an actual nerve 0.7 mm thick would in fact take only 54 seconds to reach the same saturation level. Hence, the BM is also highly vascularized, being the blood vessels responsible for an in situ delivery of important metabolic reagents and removal of its products.

The potential of bioreactors to mimic such structure is demonstrated by their capacity to support high cell densities in relatively small volumes, whilst the scaling-up of the design, usually related to mass transfer limitations, will depend on the chosen type of bioreactor. Traditionally, culture of stem cells is performed on flat two-dimensional (2D) surfaces that are used to support the growth of the cells (Dexter et al. 1977). Well-plates, tissue-culture flasks (T-flasks) (Mellado-Damas et al. 1999; Liu et al. 2006), and gas-permeable blood bags (Collins et al. 1998) are widely used in stem cell bioprocessing due to their simplicity, ease of handling and low cost making them the ideal choice for research screening purposes as well as to engineer simple tissues, such as skin, bone and cartilage (Bilodeau et al. 2006). However, lack of online monitoring, limitations in scaling-up due to the limited surface area available for cellular growth per volume, as well as their inability to support complex cellular growth configurations render 2D surface-limited systems of reduced applicability for biomanufacturing for clinical applications. 3D culture systems that would closely resemble the in vivo conditions by accounting for the cell-cell, cell-matrix and cell-growth factor interactions (Mantalaris et al. 2004b) are desirable in many clinically-relevant cases. Consequently, a variety of matrices, such as nylon screens (Naughton et al. 1991) as well as other natural or synthetic scaffolds, as described earlier, have been used to provide support for cellular growth, proving to be more efficient when compared to their 2D counterparts (Mantalaris et al. 2004b) and allowing the structuring of frameworks for the development of 3D constructs (Ott et al. 2008). However, 3D cultures with their increased available surface area for cellular attachment and growth, higher cell density, and ability for higher cell expansion, face increased mass transport limitations.

Static cultures, the so-called static bioreactors (Sardonini et al. 1993), in which the “ingredients”—cells, nutrients, metabolites, oxygen and other important molecules—experience mass transport that is exclusively through the process of diffusion result in an inhomogeneous environment that can support low cell densities and has a low total cell output (Panoskaltsis et al. 2005). To overcome the mass transport limitations of static cultures, bioreactors that can accommodate the dynamic culture conditions are desirable. Primarily, perfusion and stirring have been the main means for enhancing mass transport. Stirred suspension bioreactors require careful impeller design to avoid high shear stress that can damage the cells (Zandstra et al. 1994), can be operated either in batch or continuous mode, and result in at least a ten-fold increase in cell density over the traditional methods. The scaling-up is usually straightforward due to the very good mass transport achieved by stirring. However, the flow environment created by the impeller renders them unsuitable for support of 3D constructs (Nielsen 1999), although the inclusion of porous microcarrier beads has been considered and studied (Zandstra et al. 1994). Several perfusion bioreactors have been designed to achieve a low shear stress environment as well as enhanced mass transport that facilitates the supply of nutrient and the removal of metabolites, perfusion rate having to be optimised based on cell type. A flat-bed bioreactor has been developed containing grooves in the bottom, where cells could establish within the grooves and be protected from the perpendicular flow, thus allowing higher fluid velocities. This grooved-bioreactor has been used for the expansion and maintenance of colony-forming units granulocyte-macrophage (CFU-GM) progenitor cells and long-term culture initiating cells (LTC-IC) in the absence of stromal cells (Cabral 2001). Perfusion bioreactors have been automated providing continuous and automated feeding of the cultures. Aastrom Biosciences Inc. has developed a design whereby the cells are injected into a disposable cassette and grown on top of a previously established layer of stromal cells with nutrients being continually perfused to the cassette while a chamber, located just above, is filled with oxygen that diffuses to the cassette through a liquid-impervious/gas-permeable membrane (Armstrong et al. 1996; Palsson et al. 1997; Armstrong et al. 1999; Armstrong et al. 2000). The system has been used to expand bone marrow mononuclear cells and umbilical cord blood cells for clinical application. None of these bioreactors, however, are able to accommodate 3D growth producing constructs required in many tissue engineering applications.

A very important parameter in the design of bioreactors, as mentioned above, is the shear stresses cells will experience (King et al. 2007). Shear stress is defined as the force exerted over the cells due to the flow of the media (Chen et al. 2006), and a low rate has been described to result in cell clumping on aggregate and embryoid body cultures (leading to lower mass transport to the cells) (King et al. 2007), while high rates could be deleterious for the cells. Different cell types have different sensitivities/necessities in terms of the shear stress rate. Thus, an optimal fluid velocity promoting the proper shear stress for the cell type being cultured is crucial. As an example, mammary epithelial stem cells aggregate cultures have an optimal shear stress which is approximate to the physiological value of 2 dyn·cm-² (King et al. 2007), while endothelial cells can support higher rates, in the order of 20-30 dyn·cm⁻² (Sarkar et al. 2007). On the other hand, it has been described that the mechanical stimuli promoted this way can be beneficial for certain cell types: shear stresses in the order of magnitude of 15 dyn·cm⁻² have promoted differentiation of embryonic stem cells towards the lineage of endothelial cells, when compared to static controls (Ahsan et al. 2006). Several bioreactors have been designed for promoting this scenario in vitro, by promoting controlled shear stress levels: using dynamic tension for growing and developing cardiomyocytes, mesenchymal stem cells, skeletal muscle and macrophages; compression for chondrocytes; and hydrodynamic pressure for bone cartilage (Korossis et al. 2005). Thus, optimal shear stress levels will depend largely on the cell type being grown/differentiated, as each type will have different sensitivities and/or stimulation needs (Palsson et al. 1993). Embryonic stem cells are known to be very sensitive to shear stress levels.

Several other bioreactor designs have been implemented with varying degrees of success. These included designs such as the packed or fluidized bed bioreactors (widely used for expansion of hepatocytes, cardiocytes, osteoblasts and others) (Portner et al. 2005) and the rotating wall vessel (successful in the culture of HSCs, chondrocytes, cardiac cells, various tumour cells and others), which is a suspension culture adapted to provide a lower shear stress environment (Hammond et al. 2001; Liu et al. 2006). The main differences in their designs can be associated with mass transport (addressed either by diffusion, perfusion or bubbling), shear stresses (by developing ways to enhance mass transfer without increasing flow velocity or by which mechanical stimulation can be achieved), the ability to support 3-D constructs or even the end-purpose (research vs. large-scale, cell type characteristics, etc.).

None of the prior art designs, however, have addressed the hurdles that have kept the development of an artificial mimicry of the human bone marrow as the ultimate bioreactor for the large-scale production of blood cells, which can be summarized as follows: the high costs associated with the large-scale production of these cells (certain exogenously added growth factors, essential for the use of animal-derived serum-free environment in order to comply with Good Manufacture Practice (GMP), have costs that can reach 70,000 US$ per mg); mass transfer limitations to nourish and renew 3D structures in a large-scale scenario; and the continuous production and selective collection of mature functional cells free from potentially tumor-inducing undifferentiated stem cells, also to comply with GMP.

An interesting field that has seen growing interest in the last few decades is the development of hollow fiber bioreactors for applications that require higher surface area for contact between two different streams and the delivery of molecules. This particular design is able to provide a low shear stress environment with still enhanced mass transport properties. Such bioreactors contain a number of hollow fibres, which are responsible for carrying nutrients and oxygen to the cells, by diffusing through the selective hollow fibre membrane, thus avoiding the shear stresses caused by perfusion. At the same time, the inclusion of membrane technology in these designs highly increases the surface area per volume available for cell growth (over 350 times that of a normal T-flask), thus allowing higher cell densities, whilst still promoting efficient mass transfer of nutrients, oxygen and other important signalling molecules.

Comparing the architecture of a hollow fiber bioreactor with the highly vascularized BM, one can immediately establish a striking resemblance between both structures. On the other hand, the adaptation of the three-dimensional structure in the bone marrow with a localized mass transport of important molecules to renew the HIM, using blood vessels, can definitely be seen as an evolutional advantage as it also offers the possibility for the promotion of a heterogeneous microenvironment that is essential to promote optimal conditions to an array of different cells—with different nutrient necessities—that coexist in the bone marrow. From the engineering point of view, this association also offers enormous advantages, as it helps to tackle several problems related to scaling-up designs as concerns localized mass transport and especially in the design of new and improved bioreactors for regenerative medicine: a synthetically “vascularized” bioreactor, or a hollow-fiber bioreactor.

The human bone marrow, as shown previously, can in fact accommodate this three-dimensional growth of cells, while promoting a healthy level of nutrients and oxygen to the cells through its vascular network. In vitro, to achieve such high ratios of cells per volume (the bone marrow can accommodate up to 5×10⁸ cells·mL⁻¹)¹⁰, the inclusion of hollow tubular structures made of porous materials, within which nutrients can be perfused, on a 3D structure of cells is a necessary condition. In fact, this design can accommodate at least up to 2×10⁸ cells·mL⁻¹, a value still under the efficiency of the in vivo example, but promising in terms of future developments.

The idea of growing cells in hollow fiber bioreactors was introduced over thirty years ago. Nowadays, this design has been widely used in the production of several different proteins from mammalian cells, for the production of cells and in tissue engineering applications, such as bioartificial organs. As shown before, the possibility of maintaining these systems at near tissue densities can result in an increased per cell productivity, making high concentrations of both products and cells available. Cells are typically inoculated outside the fibers in the extracapillary (EC) space; medium is circulated from a reservoir, through the fiber intracapillary (IC) space, and returned to the reservoir afterwards. The semi-permeable fiber membrane is usually characterized as ultrafiltrative (molecular weight cutoff of 10-100 kDa) or microporous (0.1-0.2 μm pores). These systems, however, have been usually granted unsuitable for the particular application of expansion of stem cells mostly due to the difficulties of prior art designs in allowing the harvesting of the final product and hence incrementing the difficulty on their use and application.

Hollow fibre bioreactors have also been shown, due to their resemblance to the capillary network in vivo, to deliver nutrients and other required molecules in a much faster, efficient and reliable fashion than any other design developed up to now. Their high surface area to occupied volume ratio allows the delivery of these molecules, and that is particularly true in cases where the overall volume does matter, such as in attempts for scaling-up.

As described before, the culture of cells in 3D structures brings several advantages, the most obvious being the fact that this conformation resembles their in vivo environment. Another challenge, however, is to create a scaffold (that can be of either natural or synthetic nature) around these hollow fibre membranes in order to create this 3D structure onto which cells can attach and proliferate.

Prior art in the production of scaffolds has involved different techniques, such as polymerization in-situ, thermally-induced phase separation (TIPS), etc. TIPS is one of the most widely used methods for the preparation of medical-grade scaffolds that are biocompatible and promote growth and proliferation of cells. One of the greatest advantages of this method, when compared to other alternatives, is that it allows the preparation of scaffolds made of virtually any chosen material, provided that a volatile solvent within which they are soluble but chemically stable exists. Moreover, this method doesn't require the addition of eventually toxic molecules, such as initiators/promoters of polymerization in other techniques, whilst the solvent used can be totally removed from the scaffold by means of vacuum extraction and recycled for another use, also a more environmentally friendly technique.

Another advantage of the use of this method is that it allows shaping the final structure of the scaffold towards a previously made mould, as its protocol involves the liquefaction of the polymer and only afterwards its solidification. However, the hollow fibre material that will be receiving this dope solution has to be engineered to sustain the harsh conditions of the whole scaffolding procedure: low temperatures, solvent-resistant and dryness by vacuum. The present inventors have expertise in the development of solvent-resistant materials for applications in nanofiltration (also known as organic solvent nanofiltration, or OSN). This includes the development of organic solvent-resistant materials, such as polyimide, polyacrylonitrile or methods to make them chemically more stable such as through chemical crosslinking. Embodiments of the present invention disclose the use of such organic-solvent resistant materials, whether of organic or inorganic nature, chemically cross-linked or not, or even any other method that grants the material a partial to high resistance to the dissolution or any phase change promoted by the solvent used in the preparation of the scaffold, for the in situ production of a polymeric foam around them.

Other methods of scaffolding around previously disposed membranes arranged according to the final intended design, could involve other techniques that have the similar principle of fluidization of the polymeric scaffold followed by its solidification, however less disruptive to the membranes and hence further opening the available range of materials that could be used. These, also disclosed herein, could involve but are not limited to plasma treatment or supercritical solvents such as water or carbon dioxide, etc.

### BRIEF SUMMARY OF THE DISCLOSURE

In accordance with the present invention there is provided a bioreactor for the formation of mature blood cells from haematopoietic stem cells, the bioreactor comprising a first zone and a second zone, wherein the first zone and the second zone are separated by a first membrane, wherein the first membrane allows the preferential passage of red blood cells relative to the haematopoietic stem cells and their other progeny excluding red blood cells, wherein the first membrane is formed by at least a separating layer and a porous layer, where the porous layer is in contact with the first zone, such that the haematopoietic stem cells can be grown in the porous layer.

The bioreactor may comprise a third zone, wherein the first zone and the third zone are separated by a second membrane, and the second membrane allows the passage of nutrients from the third zone to the first zone and, optionally, the passage of metabolites of the cells from the first zone to the third zone, while, preferably, substantially preventing the passage of growth factors from the first zone to the third zone.

Generally, the porous layer has a three-dimensional shape (in the sense that it is not substantially laminar). The first membrane and/or the second membrane may be located within the three-dimensional shape.

In the presently preferred embodiment, the first membrane and/or the second membrane is in the form of a hollow fibre.

Viewed from a further aspect, the invention provides a bioreactor comprising a first zone defined by a porous layer having a three-dimensional shape, a second zone defined by a first membrane in the form of a hollow fibre located within the porous layer, and a third zone defined by a second membrane in the form of a hollow fibre located within the porous layer, whereby the first zone and the second zone are separated by the first membrane and the first zone and the third zone are separated by the second membrane, and wherein the porosity of the first membrane is greater than the porosity of the second membrane, such that the second membrane will retain cells or molecules within the first zone that will pass through the first membrane.

The bioreactor may comprise a recirculation circuit arranged to recirculate fluid through the third zone.

The porous layer may be a polymeric scaffold. The porous layer may be composed of at least one of the following polymers: polyurethane, poly (L-lactic-co-glycolic acid), poly (methylmethacrylate), poly (D, L-lactate), poly (caprolactone), polystyrene and derivatives thereof. Other materials are possible. For example, the porous layer may be a hydrogel. The porous layer may be a foam. The porous layer may be formed in situ about the first membrane and/or the second membrane.

The first membrane or the second membrane may be inorganic, preferably being formed of alumina oxide, titania oxide, zirconia oxide, glassy materials, or derivatives thereof.

The first membrane or the second membrane may be organic, preferably being formed of polyacrylonitrile, polyimide, polyamide, polyurethane, poly (L-lactic-co-glycolic acid), poly (methylmethacrylate), poly (D, L-lactate), poly (caprolactone), polystyrene, polyether ether ketone, polyethersulphone, polyvinylidene fluoride, or derivatives thereof.

In one embodiment, the first membrane is inorganic and the second membrane is organic.

The porous layer may have a controlled porosity from 60 to 100%. The porous layer may have a pore size distribution ranging between 0.1 μm to 1000 μm. The first membrane may have a maximum mean pore size ranging between 0.1 and 30 μm, more preferably between 0.2 and 20 μm and even more preferably between 0.5 and 5 μm.

The first membrane or the second membrane may be chemically modified, using hydrolysis or chemical crosslinking, conferring the first membrane improved resistance to organic solvents (e.g., dimethylformamide, dioxane, dimethylcarbonate, acetone and the like). The first membrane or the second membrane may have a chemical structure that is resistant to degradation for a period of at least 30 minutes by at least one organic solvent (e.g., dimethylformamide, dioxane, dimethylcarbonate, acetone and the like).

The surface of the second membrane may be modified by annealing at temperatures between 10° C. and 300° C. The surface of the second membrane may be modified by hydrolysis at temperatures between 10° C. and 200° C. to render it more hydrophilic.

The second membrane may have a molecular weight cut-off ranging between 1,000 and 30,000 Da, more preferably between 2,000 and 25,000 and even more preferably between 3,000 and 20,000.

The second membrane may be modified in order to reject by at least 10% at least one cytokine. The first membrane, the second membrane and/or the porous layer may be modified in order to attach growth factors that are known to promote angiogenesis. The first membrane and/or the second membrane may be modified in order to attach cytokines such as vascular endothelial growth factor (VEGF). The first membrane and/or the second membrane may be modified in order to attach peptides. The second membrane may be modified in order to attach cytokines that are known to promote angiogenesis. The first and/or second membranes may be modified to incorporate bioactive signals for the culture of endothelial cells. The first and/or second membranes may be modified to inhibit cell growth. The porous layer may be modified in order to render it biomimetic to include growth factors or bioactive signals. A bioreactor as claimed in any preceding claim, wherein said porous layer is modified in order to attach growth factors, such as vascular endothelial growth factor (VEGF).

Viewed from a yet further aspect the invention relates to a process for the formation of mature blood cells incorporating a bioreactor as described herein, wherein the first zone contains a mixture of cells including haematopoietic stem cells and their progeny including red blood cells, wherein the first membrane allows the preferential passage of red blood cells relative to the haematopoietic stem cells and their other progeny excluding red blood cells, wherein the porous layer is in contact with the first zone, such that the haematopoietic stem cells grow in the porous layer.

The bioreactor may have a third zone, wherein the second membrane allows the passage of nutrients from the third zone to the first zone and the passage of metabolites of the cells from the first zone to the third zone, while substantially preventing the passage of growth factors from the first zone to the third zone.

A cytokine-free environment may be used. A serum-free environment may be used. One or more cytokines may be used. Human or animal-derived serum may be used.

The first membrane may be used to provide cytokines, nutrients, oxygen and any other important molecules for the metabolic and functional activity of the cells being grown. The first membrane may be used to selectively harvest mature or nearly-mature blood cells from the first zone into the second zone, which optionally are afterwards collected in a sterile container. The second membrane may be used to provide small molecules, such as nutrients to the first zone and to remove small molecules such as cellular metabolites from the first zone.

Angiogenesis may be promoted within the porous layer in order to further enhance the transport properties of the same.

The invention extends to the use of the bioreactor for the culture of human haematopoietic stem cells with a view to the expansion of stem cells as well as the production of progenitors, precursors and mature haematopoietic stem cells.

The invention extends to the use of the bioreactor for the culture of human stem cells for the expansion of stem cells as well as the production of progenitors, precursors and mature cells derived from them.

The invention extends to the use of the bioreactor for culturing haematopoietic stem cells using growth factors in the third zone. The flow rate of the second zone may be higher than the flow rate of the third zone by a factor of 1 to 100.

The invention extends to the use of the bioreactor for culturing stem cells using growth factors by recycling the growth factors in the third zone and, optionally, renewing the second zone.

The invention extends to the use of the bioreactor for the production of human red blood cells from a source of stem cells, such as umbilical cord blood stem cells, induced pluripotent stem cells, embryonic stem cells, bone marrow stem cells and peripheral blood stem cells, or other stem cells.

The invention extends to the use of the bioreactor for culturing stem cells using an oxygen concentration in the first zone ranging from 1% to 21%.

The first membrane may be used to selectively harvest cells from the first zone into the second zone.

The invention extends to the use of the bioreactor for the production of human platelets from a source of stem cells, such as umbilical cord blood stem cells, induced pluripotent stem cells, embryonic stem cells, bone marrow stem cells and peripheral blood stem cells or other stem cells.

The invention extends to the use of the bioreactor for the production of human white blood cells from a source of stem cells, such as but umbilical cord blood stem cells, induced pluripotent stem cells, embryonic stem cells, bone marrow stem cells and peripheral blood stem cells or other stem cells.

The invention extends to the use of the bioreactor for the culture of human leukemic stem cells from either immortalized cell lines or harvested from human patients.

There is disclosed herein a method for making membranes that selectively allow the passage of growth factors and other important biological molecules; a method for selectively allowing the transport of cells; a method for constructing hollow fibres out of membranes; a method for producing three-dimensional scaffolds around hollow fibre bioreactors; a method for producing a three-dimensional hollow fibre bioreactor; a method for developing a three-dimensional hollow fibre bioreactor with two or more different types of hollow fibre membranes for the selective transport of small molecules such as nutrients (glucose, oxygen, etc.) and metabolites (lactate, carbon dioxide, etc.) while preventing the crossing of large molecules (such as growth factors); a method for developing a three-dimensional hollow fibre bioreactor with two or more different types of hollow fibre membranes for the selective transport of large biological molecules such as growth factors; a method for culturing haematopoietic stem cells using exogeneously added growth factors under reduced costs by recycling the expensive cytokines and having one or more types of designed hollow fibres to deliver nutrients while preventing the loss of these molecules; a method for the harvesting of cells from within three-dimensional structures; a method for using the three-dimensional hollow fibre bioreactor for the culture of human haematopoietic stem cells with a view to the expansion of stem cells as well as the production of progenitors, precursors and mature haematopoietic stem cells; a method for producing three-dimensional scaffolds around hollow fibres with ranging porosity, pore size, and interconnectivity in order to mimic the human bone marrow; a method for modifying the three-dimensional scaffold in order to render it biomimetic to include growth factors or bioactive signals; a method for modifying the hollow fibre membranes to incorporate bioactive signals for the culture of endothelial cells; a method for promoting angiogenesis within the three-dimensional scaffold for further enhancement of the transport properties of the same; and a method for treating the surface of the hollow fibre membranes for inhibiting cell growth.

### BRIEF DESCRIPTION OF THE DRAWINGS

Embodiments of the invention are further described hereinafter with reference to the accompanying drawings, in which:

FIG. 1 is a schematic representation of a bioreactor according to an embodiment of the invention;

FIG. 2 is a further schematic representation of a bioreactor according to an embodiment of the invention;

FIG. 3 is a schematic representation of a bioreactor configuration according to an embodiment of the invention;

FIG. 4 is a schematic representation of a bioreactor according to a further embodiment of the invention;

FIG. 5 shows two scanning electron microscope (SEM) picture of two hollow fibres for use in embodiments of the invention;

FIG. 6 is an (SEM) picture of a scaffold structure for use in embodiments of the invention;

FIG. 7 is a representation of a sintering profile for a ceramic hollow fibre for use in embodiments of the invention;

FIG. 8 is an SEM picture of a ceramic hollow fibre for use in embodiments of the invention;

FIG. 9 represents the distribution of pore size in a ceramic hollow fibre for use in embodiments of the invention;

FIG. 10 represents the distribution of pore size in a scaffold for use in embodiments of the invention;

FIG. 11 represents the effect of processing on the diffusivity of a hollow fibre;

FIGS. 12 to 16 represent the results of examples of use of a bioreactor according to the invention;

FIG. 17 is an SEM picture of a ceramic hollow fibre showing the transport of red blood cells; and

FIGS. 18 to 19 represent the results of examples of use of a bioreactor according to the invention.

### DETAILED DESCRIPTION

In broad terms, the invention provides three-dimensional hollow fibre bioreactor systems for the maintenance, expansion, differentiation and harvesting of human stem cells and their progeny.

This invention, at least in its presently preferred embodiments, relates to the design, manufacture and use of a three-dimensional (3D) bioreactor system that intimately mimics the human bone marrow in most of its architecture and functions towards the production of human blood cells. The disclosed system addresses mass transfer limitations in state-of-the-art 3D designs through the inclusion of a selectively permeable membrane system, while delivering a myriad of advantages that could allow the use of this technology to be applied in a large range of tissue engineering applications, such as: mimicking the 3D solid structure of the human bone marrow where stem cells can be grown and targeted to differentiate towards specific cellular pathways; mimicking the blood vasculature of the human bone marrow to efficiently renew the 3D porous structure by an in situ delivery of nutrients (e.g., glucose and oxygen), whilst removing toxic metabolites (e.g., lactate and carbon dioxide); allowing continuous harvesting of the cellular products being produced while relieving the space within the bioreactor for the continuing expansion of stem cells, hence increasing the productivity of the system; and highly reducing the costs of the bioprocess through the inclusion of two or more different types of membranes that will allow recycling expensive molecules while discarding toxic metabolites.

The production of the physical structure onto which cells can adhere is achieved by preparing an homogeneous liquid mixture of the desired foam (or organic polymer) into an organic solvent (e.g., when using polyurethane, 1,4-dioxane is recommended), which is then cooled down (through temperatures that range from 0 to −250° C.) and vacuum is afterwards used to remove the solvent whilst the polymer is left in place to produce a scaffold (scaffolding process). The inclusion of the design module with organic solvent resistant membranes (such as polyacrylonitrile, polyimide, polyamide, alumina or any other similar material surface-treated with cross-linking agents or not) prior to the scaffolding process allows the obtaining of membranes embedded within the foam scaffold chosen with their structure intact to perform at the desired specifications. This bioreactor, addressing mass transfer limitations of previous art designs, can therefore be used for the culture of any cell type that requires a 3D structure to sustain their growth and/or differentiation, whilst also allowing the selective harvesting of the desired product cells after targeted differentiation in situ.

This invention relates to the design and manufacture of a 3-dimensional (3D) bioreactor system, addressing mass transfer limitations with the inclusion of e.g. hollow fibre membranes (a flat-sheet configuration or any other configuration that may be appropriate can also be used). The production of the physical structure where cells can adhere onto is achieved by preparing an homogeneous liquid mixture of the desired foam (or organic polymer) into an organic solvent (e.g., when using polyurethane, 1,4-dioxane is recommended), which is then cooled down (to as low as −200° C.) and vacuum is afterwards used to remove the solvent whilst the polymer is left in place to produce a scaffold (scaffolding process). The inclusion of the design module with organic solvent resistant membranes of organic (such as such as polyacrylonitrile, polyimide, polyamide, polyurethane, poly (L-lactic-co-glycolic acid), poly (methylmethacrylate), poly (D, L-lactate), poly (caprolactone), polystyrene, polyether ether ketone, polyethersulphone, polyvinylidene fluoride or any other similar material surface-treated with cross-linking agents or not) and/or inorganic (alumina oxide, titania oxide, zirconia oxide, glassy materials or any other similar material surface-treated with cross-linking agents or not) origin prior to the scaffolding process allows obtaining hollow fibre membranes embedded within the foam scaffold chosen. This bioreactor, addressing mass transfer limitations of previous art designs, can then be used for the culture of any cell type that requires a 3D structure to sustain their growth and/or differentiation.

One of the preferred procedures of the current invention is described below.

A dope solution of the intended polymer or co-polymer to form the scaffold is prepared by dissolving said polymer or co-polymer within a solvent to a concentration up to its solubility limit and tailored for the final intended pore size. Preferred solvents are dimethylformamide, dioxane, dimethylcarbonate, acetone and others alike. Preferred scaffold material would be chosen from the following list: polyurethane, poly (L-lactic-co-glycolic acid), poly (methylmethacrylate), poly (D, L-lactate), poly (caprolactone), polystyrene and any others derived from the same.

The current design is split into three parts: 1) preparation of the hollow fibre case with the hollow fibre membranes disposed according to their intended location within the bioreactor; 2) preparation of the dope solution for the scaffold; and 3) scaffolding process followed by coating. A dope solution to form the scaffold of polyurethane (PU) is prepared with a concentration between 1 and 50%, more preferably between 2 and 48%, by dissolving stock pellets of PU in 1,4-dioxane. This solution is then dissolved within an organic solvent to a concentration up to its solubility limit and tailored for the final intended pore size. Preferred solvents are dimethylformamide, dioxane, dimethylcarbonate, acetone and others alike.

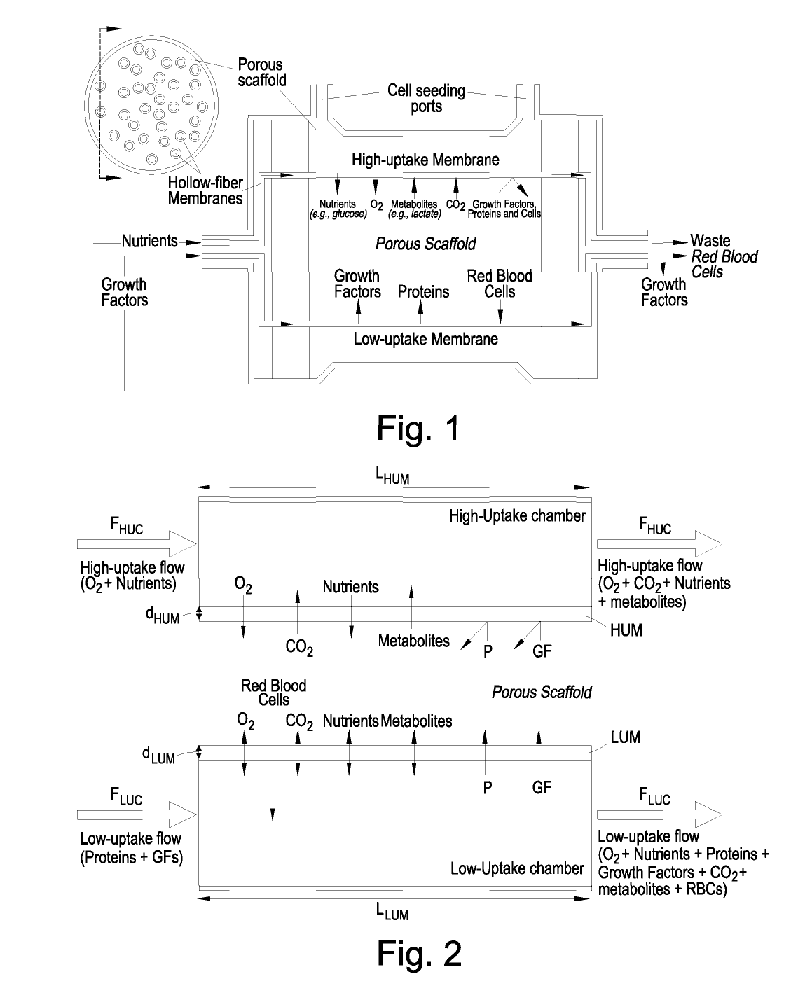

FIG. 1 shows schematically a cross-section through a bioreactor in accordance with an embodiment of the invention. FIG. 1 is a possible configuration of the proposed 3-dimensional hollow fibre bioreactor with the three zones depicted. The cross-sectional view through the porous scaffold is indicated by the dotted line and arrows in the top left of the Figure. The bioreactor comprises a plurality of hollow fiber membranes distributed in a porous scaffold. The porous scaffold is typically formed of polyurethane. As shown in FIG. 1, the porous scaffold is formed in situ within a plastics housing that is provided with cell seeding ports for injection of cells into the porous scaffold. The hollow fibre membranes run through the porous scaffold between entry and exit ports of the housing (left and right in FIG. 1). The hollow fibre membranes are of two-types: a high uptake membrane; and a low uptake membrane.

The high uptake (HU) membrane is typically formed of a polymer, such as polyacrylonitrile. The HU membrane is supplied in use with nutrients, for example glucose, which permeate through the membrane into the porous scaffold to supply the cells within the porous scaffold. The HU membrane is also capable of passing oxygen to the cells. Metabolites from the cells, such as lactate, can pass through the HU membrane to exit the bioreactor as waste. Carbon dioxide can also be absorbed through the HU membrane for removal from the porous scaffold. Larger molecules such as growth factors, proteins and cells will not pass through the HU membrane. The flow rate through the high uptake membrane is generally higher than the flow rate through the low uptake membrane and the fluid passing through the HU membrane is not recirculated but is disposed of as it exits the bioreactor.

The low uptake (LU) membrane is typically formed of an inorganic material, such as ceramic. The LU membrane is supplied in use with growth factors which permeate through the membrane into the porous scaffold to supply the cells within the porous scaffold. The LU membrane is also capable of passing proteins to the cells. Red blood cells can pass through the LU membrane for harvesting. Carbon dioxide can also be absorbed through the HU membrane for removal from the porous scaffold. The flow rate through the low uptake membrane is generally lower than the flow rate through the high uptake membrane and the fluid passing through the LU membrane is recirculated back into the bioreactor, as shown in FIG. 1.

FIG. 2 is a further schematic diagram of the bioreactor configuration of FIG. 1, as a schematic of the molecular/cellular exchanges between the three zones in a possible configuration of the proposed three-dimensional hollow fibre bioreactor system, showing some of the important parameters regarding the mass transport across them.

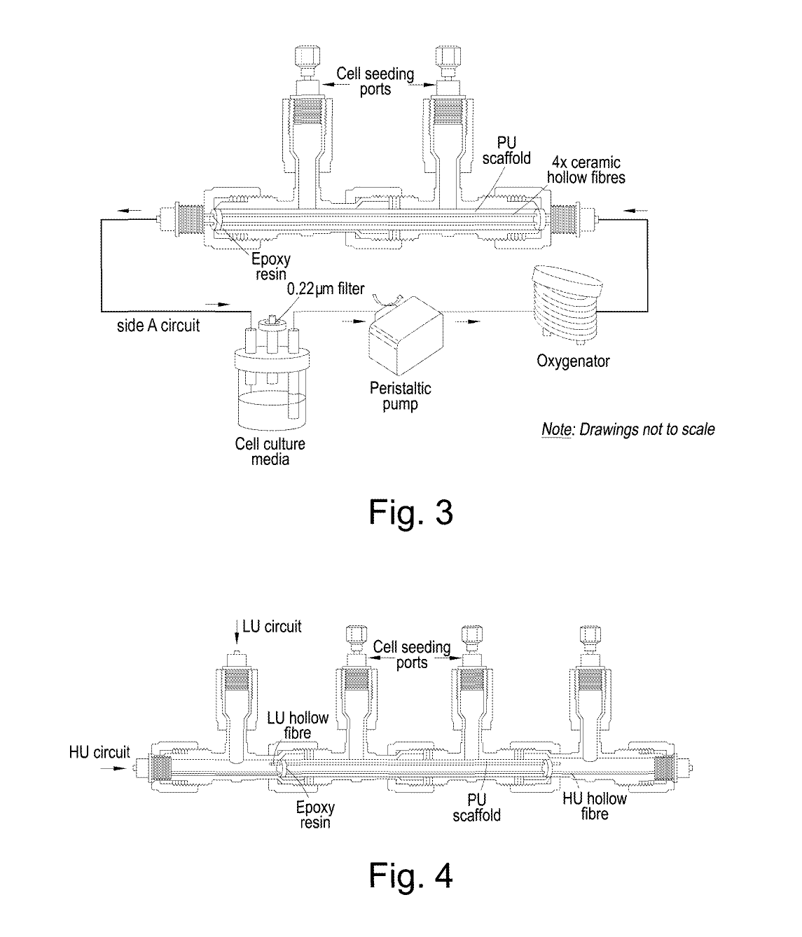

FIG. 3 is a schematic picture of the experimental setup for the culture of cells in a single-type hollow fibre bioreactor. In this case, only one type of hollow fibre membrane is used. Cells are injected through the cell seeding ports, which are then closed and maintained sealed throughout the experiment time, whilst media is continuously fed to the bioreactor through the lumen of the fibres (depicted by the arrows around the media circuit). The polyurethane scaffold, foamed around the ceramic hollow fibres, and post-coated with collagen type I, provides the physical structure where cells can attach and proliferate, while the hollow fibres continuously renew the micro-environmental niche by providing nutrients and other important molecules and removing metabolites. Media is re-circulated in the lumen of the ceramic hollow fibres and fully replaced every seven days.

For permeability testing, two different flow streams are used: within fibers (flow indicted by the arrows) and outside the fibers (flow introduced through the cell seeding ports). The flow through the cell seeding ports is also used to insert the scaffolding solution around the fibers, as well as to inject the cells afterwards. A polyurethane scaffold, foamed around the hollow fibres, and post-coated with collagen provides the physical structure where cells can attach and proliferate while having the microenvironmental niche being continuously renewed by the perfusion of nutrients through the hollow fibres.

FIG. 4 shows the dual hollow fibre design of the bioreactor (corresponding to the configuration of FIGS. 1 and 2), with both hollow fibre types depicted: the lowermost fibre in the figure is the high uptake (HU) ultraporous polyacrylonitrile hollow fibre for exchange of small metabolic molecules; the uppermost (shorter) fibre is the low uptake (LU) microporous ceramic hollow fibre for delivery of large molecules such as cytokines and harvesting of red blood cells. Also depicted in the figure are the access ports for each hollow fibre stream that enters the bioreactor, which in the centre are surrounded by a porous structure made of polyurethane (PU) to support the adhesion and growth of haematopoetic stem cells and their progeny.

The designed bioreactor allows the inclusion of two different types of hollow fibres, for the delivery and use of two different streams of molecules. FIG. 4 shows the solution to the challenge of the inclusion of two different streams feeding the bioreactor by means of access ports for each fibre. The inclusion of a polyurethane (PU) scaffold surrounding the hollow fibres at the centre of the bioreactor, where cells are injected through the two central ports in the diagram, allows the provision of a solid structure where cells can reside, proliferate and produce red blood cells which are afterwards harvested through side B (LU side) of the design.

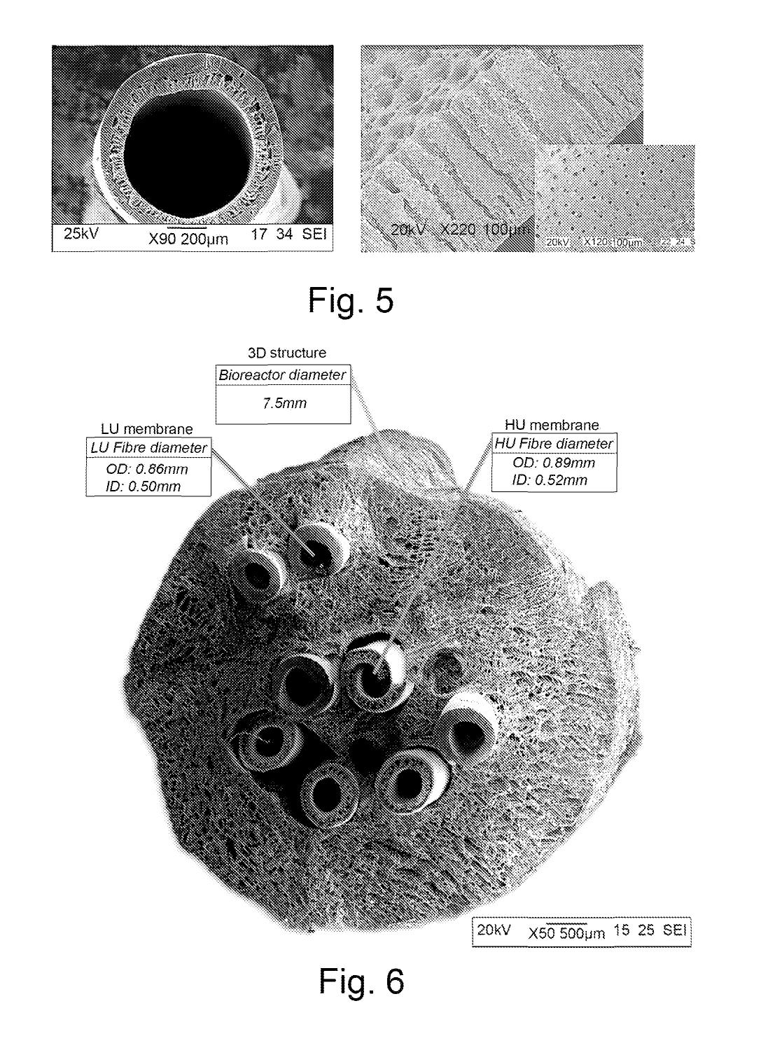

FIG. 5 shows the two different types of hollow fibres used in the bioreactor. On the left of FIG. 5 is the polymeric hollow fibre which prevents waste of growth factors, delivers nutrients and removes metabolites and is a single-pass fibre in the sense that the media is not recirculated through the fibre. In the bioreactor the flow rate through the polymeric (polyacrylonitrile) fibre is relatively high. On the right of FIG. 5 is the ceramic hollow fibre which delivers serum and/or growth factors and continuously harvests red blood cells. In the bioreactor the flow rate through the ceramic fibre is relatively low and media is recirculated through the fibre.

FIG. 6 shows the HU membrane fibres and the LU membrane fibres in situ in the porous scaffold (“3D structure”).

### Preparation of High Uptake Membranes

Membranes that selectively allow the passage of growth factors and other important biological molecules (for the fast-flow, high uptake circuit) can be made according to the following method. Polyacrylonitrile (PAN) membranes are one of the best candidates as ultrafiltrative hollow fiber membranes, allowing the exchange of nutrients and metabolites with the feeding stream whilst preventing the crossing of large protein molecules. In general, these membranes should present the following specifications:
a) Reject completely a model protein (α-lactalbumin, MW=14,200 Da, PI=4.2-4.5);
b) Allow crossing of nutrients (glucose) and metabolites (lactate);
c) Be scaffold process-resistant (solvent contact, low temperatures and dryness);
d) Flexible to be adapted to the bioreactor configuration; and
e) Made of a biocompatible material.

Previous experiments using PAN membranes, but in flat-sheet configuration, had suggested that a molecule of 10 kDa size (PEG 10 kDa) could be rejected by a membrane composed of 15 wt % in DMF, see table below which shows rejection results of three different surface-treated PAN membranes (15 wt % in DMF) to PEG 10 kDa, after dead-end cell filtration at 10 bar.

| Membrane | Rejection to PEG 10 kDa |
|---|---|
| 2. PAN without surface treatment | 52% |
| 3. PAN hydrolysed (1M NaOH at | 96.1% |
| 55° C. for 30 mins) | |
| 4. PAN hydrolyzed and cross-linked | 95.9% |

However, those experiments had been made using a dead-end cell and at 10 bar, conditions very different from those required for the bioreactor, which has a hollow fiber configuration without any applied pressure and with molecular exchange by diffusion only.

It was identified as possible that the collapse of the PAN structural matrix due to the high pressure applied in the flat sheet configuration could be responsible for the increase of the rejection of the model protein. For this reason, the hollow fiber membranes were annealed by boiling them at high temperatures to promote a higher stabilization of the PAN matrix structure that can mimic the behaviour observed when high pressure is applied in the flat-sheet configuration—reducing its pore size or matrix structure to prevent the crossing of large molecules, in the size range of the model protein α-lactalbumin.

All samples were annealed in water at 96° C. for 10 s. To perform these experiments, four bioreactors in parallel were used, each composed of a different surface treatment of the PAN hollow fibers: only annealing at 96° C.; annealing at 96° C. followed by hydrolysis at 80° C. with 1M NaOH; annealing at 96° C. followed by hydrolysis at 60° C. with 2M NaOH; annealing at 96° C. followed by hydrolysis at 60° C. with 1M NaOH. The solutions inserted are as follows:
Side A (into the lumen of the fibres): Glucose+PBS (volume=30 mL)
Side B (outside the fibres, i.e. in the scaffold): α-lactalbumin+PBS (volume=10 mL)
Flow rates: app. 1.7 mL·min⁻¹

The results obtained are presented in the table below, which shows the permeability results of the PAN hollow fibres to both glucose (nutrient) and lactate (metabolite) molecules (all PAN membranes are 18 wt % in DMF and annealed in water at 96° C. for 10 s) (n=1). K_{OV} is the overall mass transfer coefficient; D is the diffusion coefficient through the membrane. n=2 for all experiments.

| | | 2. Annealing | 3. Annealing | 4. Annealing |
|---|---|---|---|---|
| | | and hydrolysis | and hydrolysis | and hydrolysis |
| | 1. Only | at 80° C. with | at 60° C. with | at 60° C. with |
| | annealing | 1M NaOH | 2M NaOH. | 1M NaOH. |
| K_{OV} for Lactate | 13.8 ± 2.3 | 31.4 ± 2.8 | 15.3 ± 2.8 | 28.5 ± 4.5 |
| (×10⁸ m³ · m⁻² · s⁻¹) | | | | |
| D for Lactate^{†} | 24.9 ± 4.1 | 64.7 ± 5.8 | 30.7 ± 5.6 | 64.4 ± 10.1 |
| (×10⁸ cm² · s⁻¹) | | | | |
| K_{OV} for Glucose | 16.3 ± 5.5 | 50.6 ± 4.3 | 19.5 ± 10.2 | 31.6 ± 0.1 |
| (×10⁸ m³ · m⁻² · s⁻¹) | | | | |
| D for Glucose^{††} | 29.4 ± 10.0 | 104.2 ± 8.9 | 38.9 ± 20.4 | 71.3 ± 0.2 |
| (×10⁸ cm² · s⁻¹) | | | | |
| Amount of protein | 292.4 | 146.8 | 113.2 | 190.1 |
| adsorbed after | | | | |
| 2 × 75 h (μg · cm⁻²) | | | | |
| Rejection to α-L | 80.3 | 92.5 | 93.9 | 88.8 |
| (% after 75 h) | | | | |

^{†}lactate diffusion coefficient in water (at 37° C.) = 480 × 10⁻⁸ cm² · s⁻¹
^{††}glucose diffusion coefficient in water (at 37° C.) = 924 × 10⁻⁸ cm² · s⁻¹

From these results it is possible to conclude that this process does indeed change the morphological structure of the membrane and this is confirmed by SEM images of the PAN hollow fibres. It is also possible to observe that the hydrolysis of the surface of the membrane enhances its permeability properties, by increasing the overall mass transfer coefficient of both molecules tested, i.e. glucose and lactate. In order to render PAN membranes both more hydrophilic (hence less prone to fouling by protein adsorption to their surface) and less permeable to large molecules (above 10 kDa molecular weight), a surface treatment by hydrolysis of prepared membranes is employed. SEM pictures of membranes prepared in flat-sheet and hollow fiber configuration taken before and after hydrolysis confirm the effect that this procedure has on the morphology of the membranes.

Observing SEM pictures of the PAN membranes before and after hydrolysis, it is possible to see that this surface treatment leads to a slight change in the morphology of the membranes. More specifically, it is possible to observe that the diameter of the hollow fibres reduces slightly after hydrolysis (from 1.2 mm to 0.8 mm, or 33% reduction in size). Consequently, where the hydrolysis of the fibres is carried out in situ, i.e., after having cured the bioreactor inlets with epoxy resin, the fact that the hydrolysis reduces the diameter of the fibres can cause leakage. Due to this observation, all bioreactors are prepared by treating the surface of the PAN membranes before constructing the bioreactor.

The influence of the hydrolysis of the PAN hollow fiber membranes for their diffusivity to both glucose and lactate has been measured and shows the surface hydrolysis reduces the diffusivity of glucose by 92.2% and that of lactate by 25.6%.

### Preparation of the Low Uptake Membranes

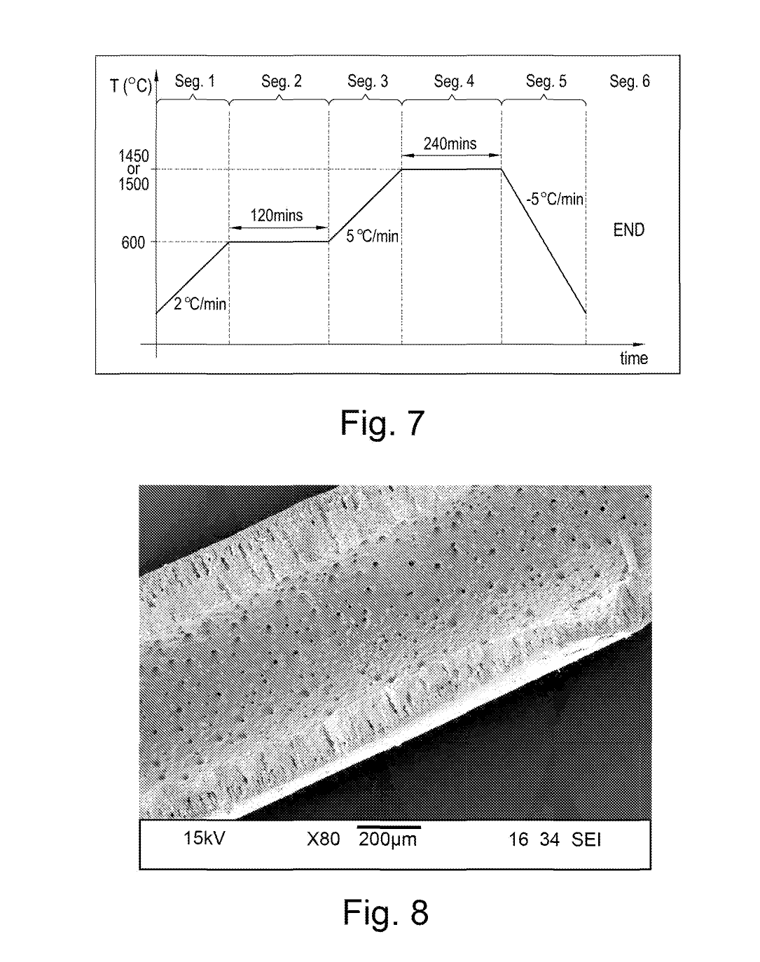

FIG. 7 summarises the sintering profile used for the inorganic hollow fibre membrane. Due to the brittleness of these fibres (probably due to their small diameter of around 800 μm), their handling during the preparation of the bioreactor sometimes leads to breaks in the fibres that force the restart of the process. An increase in the sintering temperature is expected to lead to an increase in the strength of these fibres. The table below shows a comparison of the characteristics of the hollow fibres after different sintering temperatures. The maximum pore size in the outer layer is determined by mercury displacement porosimetry.

| | | | Maximum pore |
|---|---|---|---|
| Sintering | Mechanical | Outer | size in the |
| temperature | strength | diameter | outer layer |
| (° C.) | (MPa) | (μm) | (μm) |
| 1450 | 15.04 | 793 | 3.5 |
| 1500 | 27.31 | 713 | 2.5 |

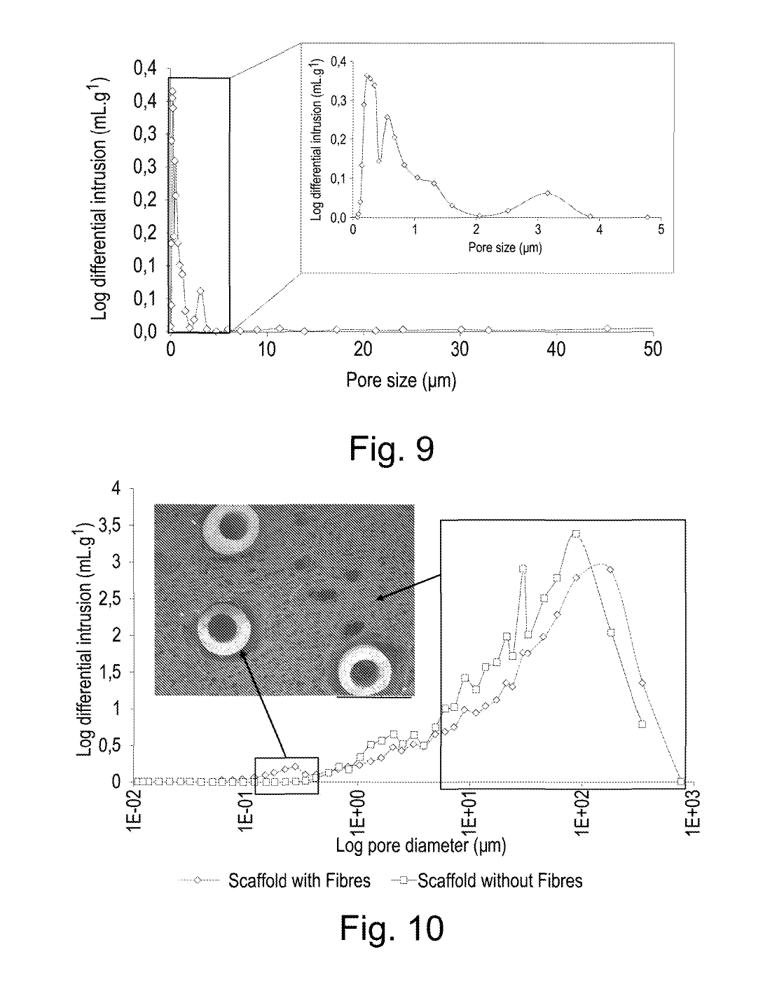

FIG. 8 is an SEM picture of the inside layer of the thin ceramic hollow fibres, obtained after sintering at 1450° C. The high porosity is clearly visible on the inside while the pore-size is reduced from the inside to the outside, the outside layer containing the smallest pores. Mercury porosimetry displacement has been used to determine the pore size distribution of the ceramic hollow fibres. SEM pictures show an increase of the pore size from the outer surface to the inner surface. Due to the fact that the mercury would flood the higher pores on the inner surface in the first place, the edges of the hollow fibres were glued with epoxy, to prevent the mercury entering through the edges. FIG. 9 shows the pore distribution (assuming pores are round, which probably is not the case) on the inner surface according to mercury porosimetry displacement analysis of the ceramic hollow fibres. The pore size measured is proportional to the pressure required to force the mercury to enter the pores, considering these are perfectly round: the larger plot shows no pores in the range above 4 μm on the outer layer of the membrane, whilst the magnification in the smaller pore-size ranges shows the pore distribution on the outer surface of the membrane to be 3.1±0.7 μm. From the figures, it is clearly seen that the biggest pores on this membrane have between 2.5 μm and 3.5 μm, which is the range required for the crossing of red blood cells.

### Preparation of the Porous Scaffold

In order to produce a three-dimensional structure around hollow fibres, there should be sufficient stability of the hollow fibre materials under the scaffolding conditions. The scaffolding protocol around the hollow fibres involves the following steps:
Dissolve polymeric scaffold material in its corresponding organic solvent (DMC for PLGA and 1,4-Dioxane for PU);
Contact the hollow fibre membranes with the dissolved polymer;
Freeze the whole bioreactor under liquid nitrogen for 2 hours (or −86° C. in the case of PU);
Transfer the bioreactor to a −25° C. bath, and remove the solvent by lyophilisation (a process that, typically, requires around 2 days) until the polymer is completely dried and free of any solvent;

A PAN membrane forms the fast-flow circuit with scaffolding. Polyacrylonitrile has very good chemical and thermal stability and has wider applicability in the ultrafiltration range. SEM pictures obtained for the PAN hollow fibres prepared before and after the scaffolding treatment show that, from the morphological point of view, the scaffolding process does not appear to influence the structure of the hollow fibres. Permeability and rejection studies of these membranes prove this. The result supports the hypothesis that this membrane can be used to design a membrane bioreactor with a scaffold around it. This 3D structure has previously been demonstrated to support the growth and viability of stem cells in culture. Hydrolysis of these fibres is an important step in making them effective as the fast-flow membrane. Their resistance to this treatment has been checked as well.

The influence of the scaffold treatment with 1,4-dioxane and freezing at −86° C. without adding polymer on the PAN hollow fiber membranes for their diffusivity to both glucose and lactate has been measured and shows the treatment reduces the diffusivity of glucose by 50.6% and that of lactate by 3.1%.

In order to determine the quality of the scaffold material and porosity for tissue engineering applications, its morphological characteristics have been determined by mercury intrusion porosimetry, using an Autopore IV 9500 apparatus (Micromeritics, UK). In order to study the pore size distribution inside the scaffold, alongside the distribution of pores when ceramic hollow fibres are present, two samples were analysed: scaffold material obtained from the HF bioreactor without fibres; scaffold material obtained from the HF bioreactor having two ceramic hollow fibres dispersed therein. The size of the samples was of around 1 cm long and 1 cm diameter. Measurements of porosity, tortuosity and total pore area are shown in the table below and the pore size distribution is shown in FIG. 10, which shows mercury displacement porosimetry of PU scaffolds produced with (0.06 g sample weight) and without (0.07 g sample weight) ceramic hollow fibres immersed.

| | | | | BET |
|---|---|---|---|---|
| | Sample | | | surface |
| | weight | | | area |
| Sample | (g) | Porosity | Tortuosity | (m² · g⁻¹) |
| PU without fibres | 0.0583 | 79.1 | 1.4058 | 230.0 |
| PU with 2 ceramic | 0.0650 | 81.7 | 0.9311 | 230.0 |
| hollow fibres immersed | | | | |

From FIG. 10 it is possible to observe that the pore size distribution of the PU scaffold produced ranges from 20-200 μm, with a pore distribution peak at around 100 μm, which renders it suitable for tissue engineering applications (there is enough space for cells to disperse and grow, as these have diameters in the range of 7-20 μm). Comparing both plots in FIG. 10, one can observe the existence of a pore distribution peak between 0.1-0.3 μm, which corresponds to the mercury intrusion into the ceramic hollow fibres (as it is absent from the scaffold without ceramic hollow fibres immersed). It is worth noting that this is not the actual pore distribution of the ceramic hollow fibres in the outer layer, as seen in previous analysis, since the mercury intrusion works from inside of the fibres to their outlet (due to the larger pores on that interface), while the larger pore size distribution of the outer layer is actually overlapped by the scaffold material.

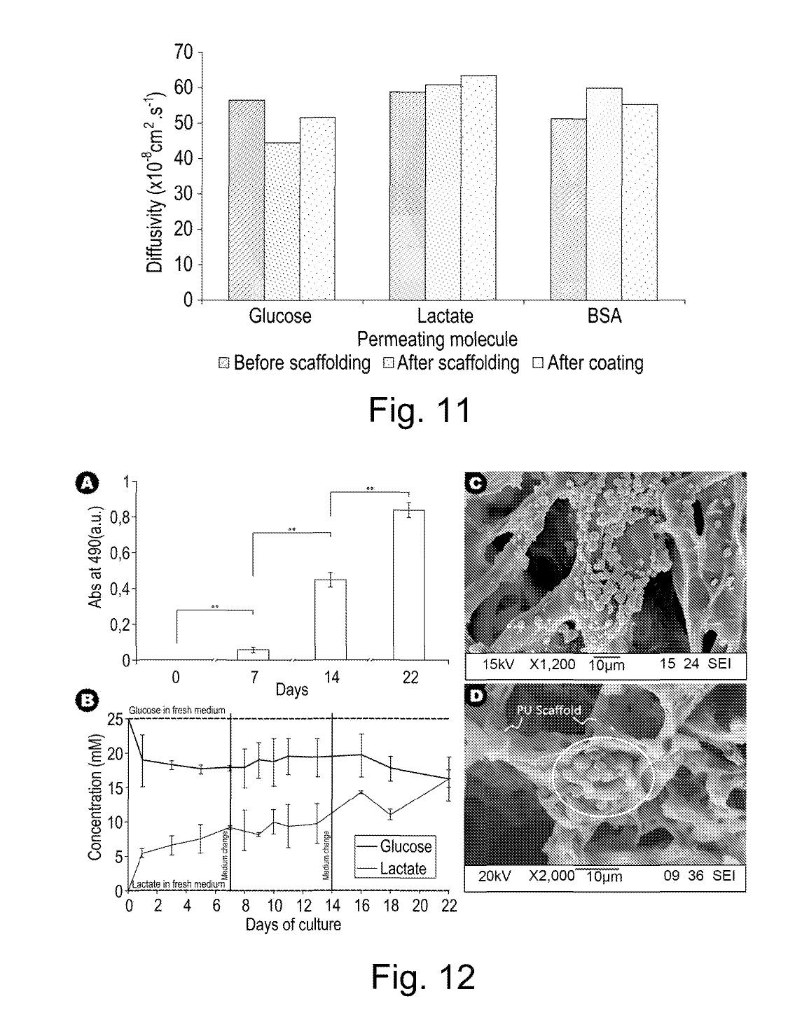

FIG. 11 summarises the diffusivities measured for the ceramic hollow fibres towards nutrients (glucose), proteins (bovine serum albumin) and metabolites (lactate). The effect of the scaffold manufacturing process (before scaffolding, after scaffolding and after protein coating of the scaffold with collagen) towards the diffusivity behaviour of the fibres is also represented. The ceramic hollow fibre membranes allow the selective mass transport of albumin as well as in the red blood cells, mimicking the body vasculature.

### Preventing Blocking of Ceramic Hollow Fibres

It has been found during the development stage that the ceramic hollow fibres could be blocked by an infiltration of polyurethane during the scaffolding process. Using SEM pictures, polyurethane (PU) scaffold was observed within the ceramic hollow fibres. To overcome this problem, the inside of the fibres were protected with guitar strings (as a suitable thin insert), in order to allow liquid to flow through the lumen of the fibres uninterrupted. In order to further assess the advantage of using this technique to prevent the blockage of the fibres, the inlet pressure of liquid being introduced into the bioreactor was measured in two different bioreactors: a 3D ceramic hollow fiber bioreactor produced without using the guitar strings; and a 3D ceramic hollow fiber bioreactor produced using the guitar strings. Using an artisanal U-tube manometer, the inlet pressures were measured for both bioreactors, at a constant flow rate of 1.7 mL·min-1 (peristaltic pump at a speed rate of 10 rpm). Without scaffolding, the inlet pressure was 6.7 mbar. Without the use of guitar strings during the scaffolding process the inlet pressure was increased to more than 19.6 mbar. When the guitar strings were used during the scaffolding process the inlet pressure subsequently returned to 6.7 mbar. The protection using guitar strings is shown to have worked in maintaining the lumen of the hollow fibres free from the polymeric foam.

### Example 1

### Culture of Stem Cells in a Single-Type HF Bioreactor in a Cytokine-Free Environment (Establishment of Normal Haematopoiesis)

Mononuclear cells (MNCs) isolated from umbilical cord-blood (UCB) were cultured in a single-type HF bioreactor (see FIG. 3), in the absence of any exogenously-added cytokines. This design of bioreactor is composed of four ceramic hollow fibres immersed within a polyurethane scaffold coated with collagen type I.

A volume of 5 mL of cell culture medium containing UCB-MNCs were seeded onto the PU-coated scaffold at a final density of 1.6×10⁷ cells·mL⁻¹. A total volume of 400 mL of medium composed of IMDM with 30% FBS and 1% pen/strep was perfused through the lumen of the ceramic hollow fibres in recirculation mode and fully replaced every 7 days of culture. The flow rate was 6.7 mL·h⁻¹. The cells were cultured over a period of 22 days in a cytokine-free environment.

Evaluation of the capacity of the single-type HF bioreactor to sustain cellular growth, by culturing MNCs is shown in FIG. 12. The cellular growth profile was measured by MTS (n=3, N=3), as shown in FIG. 12A (**p<0.01). The light absorbance measured at each time point, proportional to the number of viable cells, was normalized to the day 0 value (collected after seeding the scaffold, waiting 1 h in the incubator, and immediately terminating the bioreactor). As shown in FIG. 12B, the profile of glucose and lactate over time in cell culture medium was measured at the outlet of the bioreactor (black vertical lines represent full medium exchange) (n=2, N=3). FIGS. 12C and 12D are SEM pictures of cellular niches formed within the PU scaffold (white circle) after 22 days of serum-free culture (magnifications: ×1200 and ×2000, respectively).

The viability of the harvested cells was 73.5±14.0%, 74.5±6.1% and 67.3±9.5%, respectively, which were not statistically different (p>0.4). Representative scaffold samples from each collection day were analysed for cellular metabolism and proliferation by MTS assay, as shown in FIG. 12A. Cellular growth increased steadily throughout the culture period, with a 17.7±0.1-fold expansion of the initial cellular activity measured after 22 days of culture. The concentration profile of glucose and lactate throughout the culture period were evaluated by periodic collection of media samples from the outlet of the bioreactor, as shown in FIG. 12B. The concentration of glucose remained above 15 mM, whereas there was a steady increase of lactate throughout the 22 day culture period (reaching a final concentration of 16 mM). After 22 days, scaffold sections were observed under SEM for evaluation of the morphology of the cellular population that had established within the pores, as shown in FIG. 12C. Heterogeneous colonies of healthy hematopoietic cells were observed scattered throughout the 3D scaffolds, growing within the pores, in niche-like structures, as shown in FIG. 12D.

The bioreactor sustained long-term cellular growth with a 17.7±0.1-fold expansion in the absence of exogenous cytokines after 22 days of culture, emphasising the importance of the 3D structure provided by the PU scaffold. The concentration profiles of glucose and lactate in the 3D-HFB culture media over the 22-day period provide an indication of active cellular metabolism, with the production of lactate and consumption of glucose. Throughout the culture period, the requirements of the cell culture microenvironment in terms of the flow of these molecules were efficiently managed by the membranes in terms of removal of metabolites and provision of nutrients. The concentration of glucose throughout the culture time is above its low limit for cell growth (1 mM) and lactate is below its maximum level (22 mM) for cell growth inhibition. Due to the accumulation of metabolites in the re-circulated media, for long-term culture (beyond the period herein reported) this bioreactor could be operated in a single-pass perfusion of nutrients, therefore maintaining the levels of glucose and lactate within healthy levels.

### Example 2

### Culture of Stem Cells in a Dual-Type HF Bioreactor for the Production of Human Red Blood Cells

The designed 3D dual HF bioreactor (see FIG. 4) incorporates two different hollow fibre types, for the delivery and use of two different streams of molecules. FIG. 4 presents the schematic of this bioreactor, which integrates two different streams for feeding the bioreactor. MNCs isolated from UCB were seeded into the bioreactor and a cocktail of 100 ng·mL⁻¹ SCF and 3,000 mU·mL⁻¹ EPO were used to further potentiate the expansion of these cells (when compared to the cytokine-free environment described above) and drive the differentiation of the cells towards the enucleated RBCs.

Two bioreactors, composed of four PAN hollow fibres and four ceramic hollow fibres were prepared, according to the protocol developed for the fabrication of the bioreactors as described above. PAN hollow fibres were treated by annealing at 96° C. for 10 s followed by surface hydrolysis at 80° C. for 25 minutes with 1M NaOH, as previously described. The media circulating in each side has the following specifications:
Side A (Fast-flow circuit, within the PAN HFs)—set at 20.0 flow rate (53 mL·day⁻¹)
   IMDM serum-free+1% Pen-strep (circulated in single-step, discarding the media flowing out of the bioreactor);
Side B (Slow-flow circuit, within the ceramic HFs)—set at 15.0 flow rate (˜9 mL·day⁻¹)
   110 mL IMDM+30% FBS+1% Pen-strep+220 U of EPO (2 U·mL⁻¹)+11 μg of SCF (100 ng·mL⁻¹)—recycling 100%.

A unit of umbilical cord-blood is used to seed each bioreactor. The numbers are the following:
Bioreactor A: 11.1×10⁸ viable cells seeded (with 69% viability);
Bioreactor B: 7.4×10⁸ viable cells seeded (with 84% viability);

After 31 days of culture, inspection of the bottom of the Bioreactor A, particularly at the outlet side from the slow-flow membrane showed a deposition of cells with red coloration (typical of red blood cells) throughout the whole bottom surface of the flow; due to the slow flow there is a high deposition of cells in this area, after exiting from the bioreactor. The morphology of the blood cells produced in the dual hollow fibre bioreactors after 31 days of culture of mononuclear cells was visible.

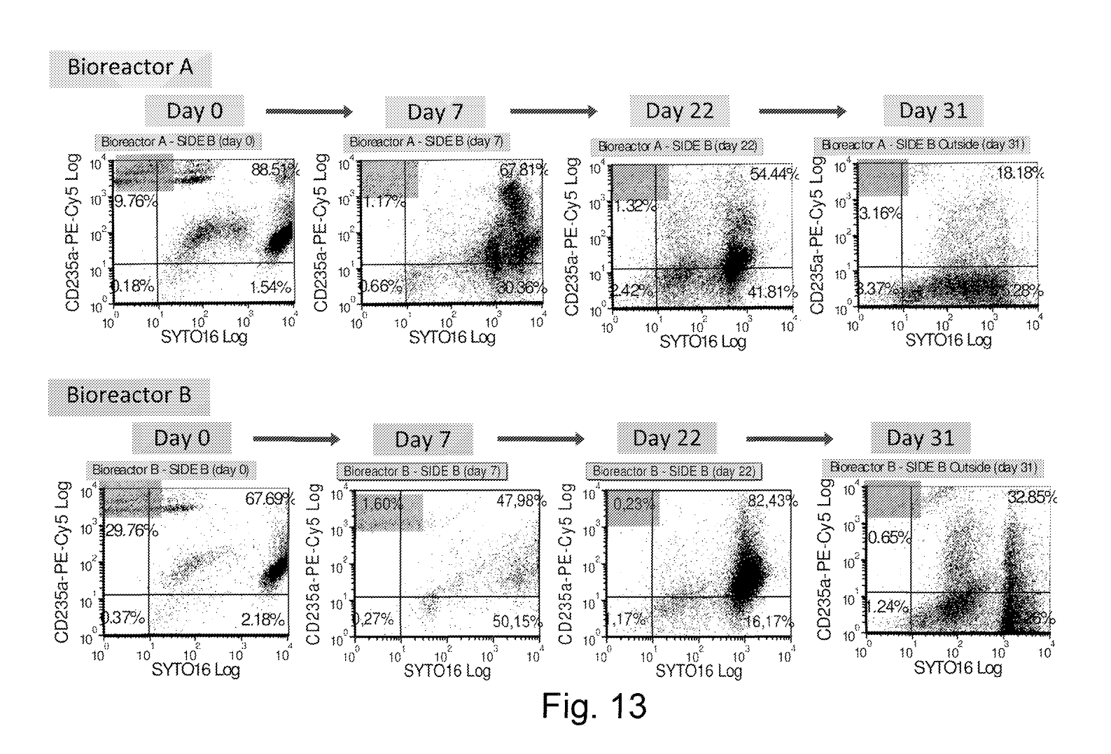

FIG. 13 summarises the flow cytometry analysis of the cells being collected along with the culture time period. The shaded bar in the top left-hand corner of each chart highlights the enucleated red blood cells, which have no nucleus (hence negatively express SYTO16) and highly express CD235a (a red blood cell surface antigen marker).

The table below is a summary of the cell numbers produced and harvested in both bioreactors (A and B) after culture of MNCs for 31 days in the 3D dual hollow-fibre bioreactor treated with 100 ng·mL-1 SCF and 2 U·mL-1 EPO.

| Number of cells | Bioreactor A | Bioreactor B |
|---|---|---|
| Seeded (×10⁸ cells) | 11.1 | 7.4 |
| Crossed the ceramic HF after | 297.8 | 2.6 |
| 31 days (×10⁸ cells) | | |
| Collected from the PU scaffold | 73.8 | 2.6 |
| after 31 days (×10⁸ cells) | | |
| Fold-expansion (considering | 33.5 | 0.4 |
| only harvested cells) | | |
| Enucleated RBCs at Day 0 | 1.1 | 2.2 |
| (×10⁸ cells) | | |
| Enucleated RBCs at Day 31 | 9.4 + 5.9 | 0.02 + 0.01 |
| (×10⁸ cells) | | |
| RBCs fold-expansion | 13.9 | 0 |

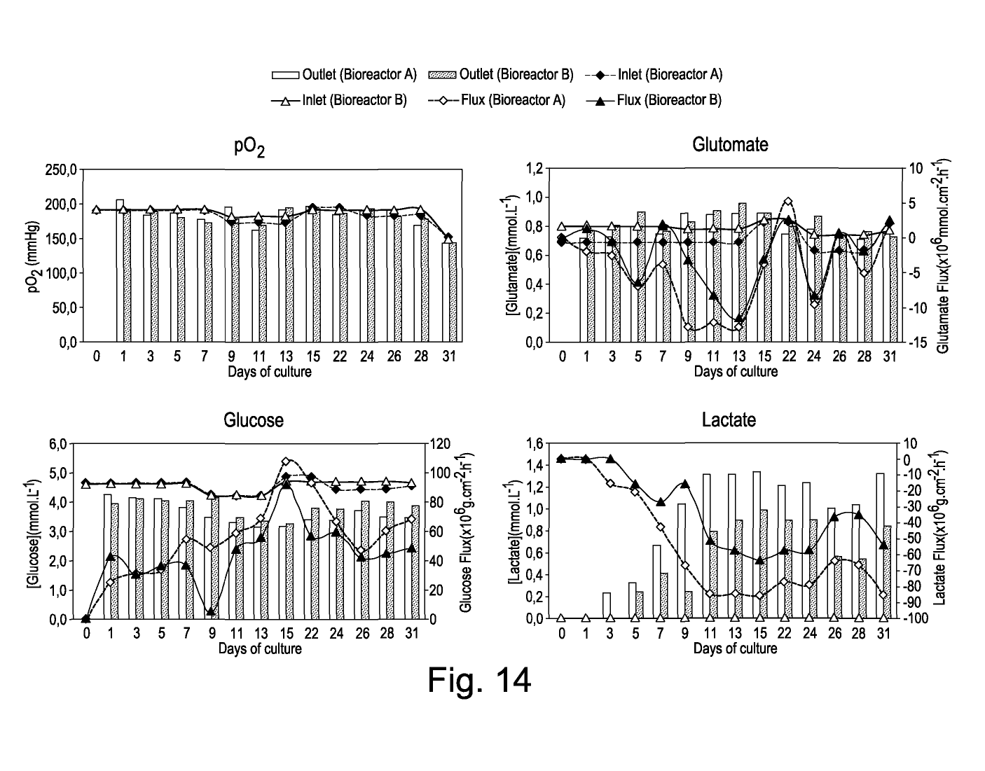

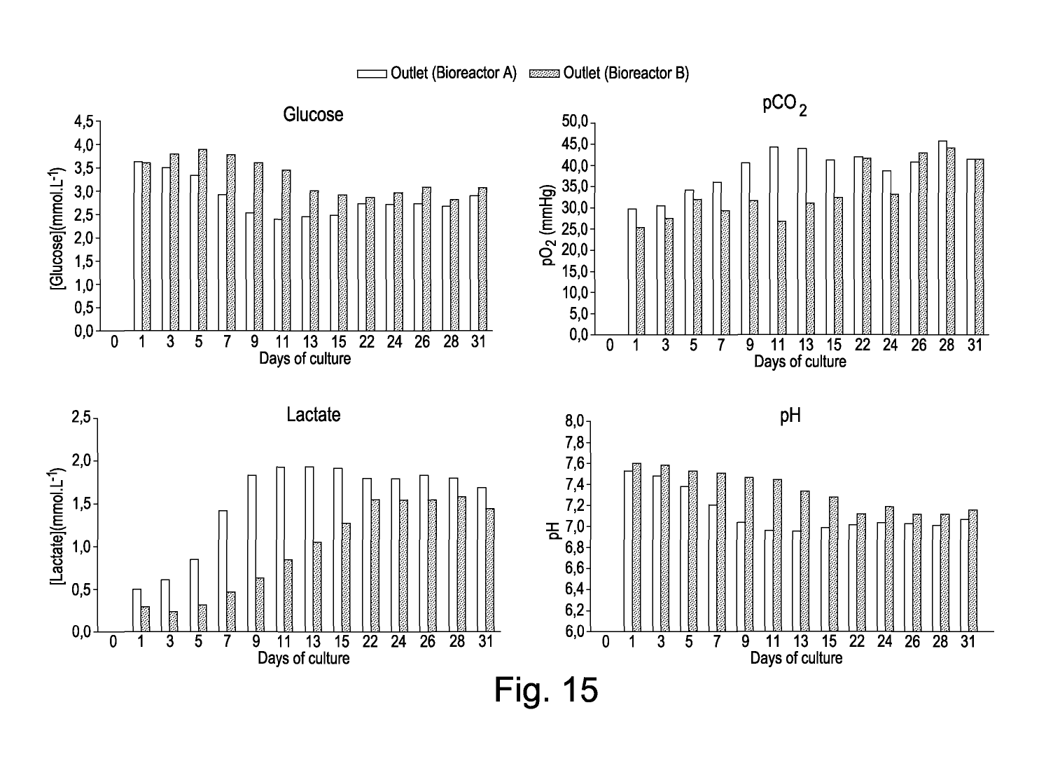

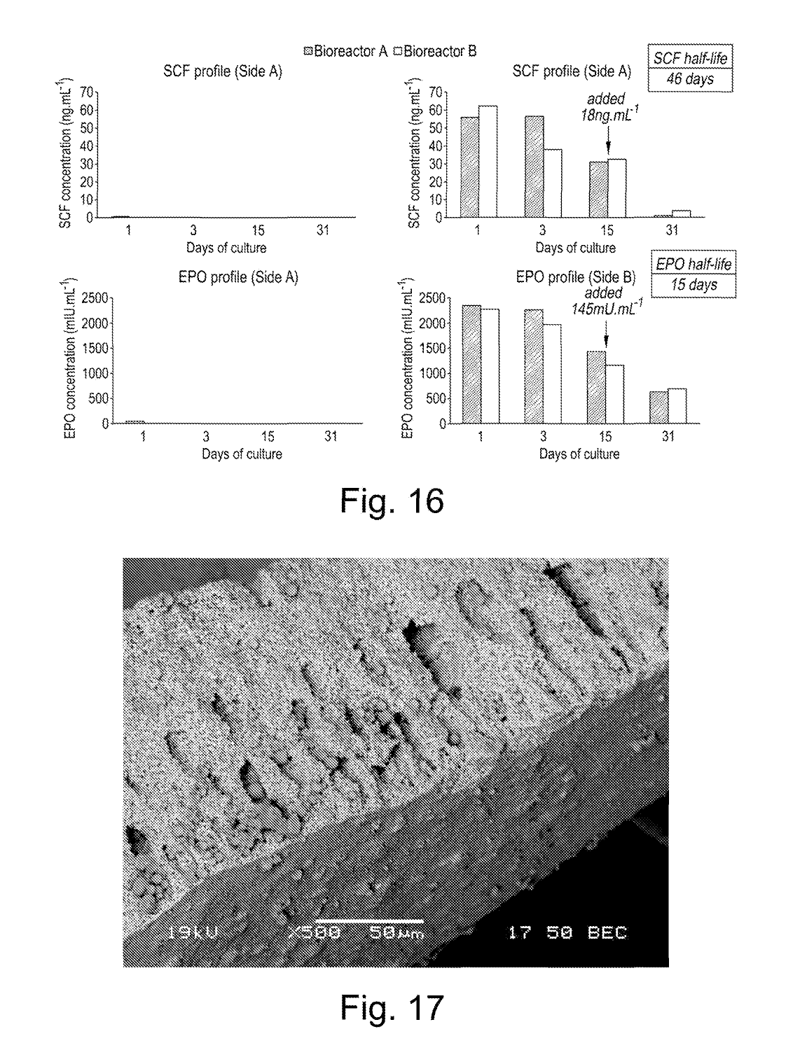

FIGS. 14 and 15 show the bioprofiler analysis of sides A (FIG. 14) and B (FIG. 15) of both bioreactors. FIG. 16 shows the growth factor analysis of sides A and B

From the analysis shown, both in terms of the bioprofiler (which provides an idea of the concentration of nutrients and metabolites within the bioreactor) and the growth factor concentrations (which allows perception of the presence of these important molecules for the survival, maintenance, proliferation and differentiation of stem cells), the following conclusions can be drawn. The fast-flow membrane is capable of providing a proper culture environment to the cells, since the molecular profile of side B (which is recirculated and not replaced) remains constant throughout the culture period, at healthy levels. Since the concentration within the fast-flow circuit is not replaced, the concentrations of molecules within it are very similar to those within the space where the cells are growing, hence proving this system is capable of providing the cells with a healthy level of nutrients and metabolites; the fast-flow membrane is also capable of preventing the waste of growth factors, since there is no visible detection of these proteins at the outlet of side A.

FIG. 17 shows red blood cells populating the ceramic hollow fiber (white arrow: direction of the flow of the red blood cells, from the outside to the inside of the hollow fiber). Several cells are seen on the outer surface of the hollow fiber trying to enter the membrane, with some of them squeezing through (SEM obtained with a backscatter filter).

### Example 3

### Culture of Leukemic Cells from Patients in 3D Scaffolds (Establishment of Abnormal Haematopoiesis)

Acute myeloid leukemic (AML) cells harvested from patients were seeded into the 3D scaffolds used on the HF bioreactor, in order to study the potential of this porous material in supporting the growth of abnormal haematopoiesis. Cells were harvested from the bone marrow of patients, following informed consent. These were then seeded onto sterile cubic scaffolds with 5×5×5 mm³ at a concentration of 2.5×10⁶ cells/scaffold (100 ml of cell suspension), placed in 24-well tissue culture plates and incubated over a maximum period of 28 days at 37° C. and 5% CO₂ with 1.5 ml Iscove's Modified Dulbecco's Medium (IMDM) with 30% fetal bovine serum (FBS) and 1% Penicillin/Streptomycin (pen/strep). Half-medium exchange was carried out every other day. Cytokines were not added at any stage of the cell culture.

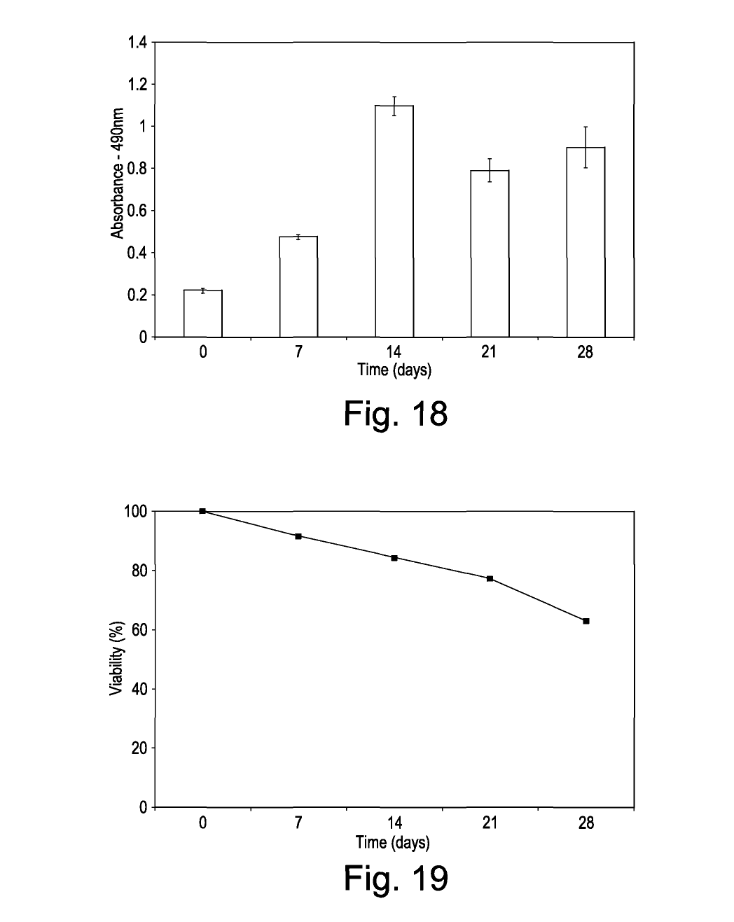

FIG. 18 shows the cellular growth profile of AML cells in the 3D scaffolds. FIG. 19 shows the cellular viability of AML cells grown in 3D scaffolds over time.

In summary, a bioreactor for the formation of mature blood cells from haematopoietic stem cells is disclosed. The bioreactor comprises a first zone and a second zone. The first zone and the second zone are separated by a first membrane. The first membrane allows the preferential passage of red blood cells relative to the haematopoietic stem cells and their other progeny excluding red blood cells. The first membrane is formed by at least a separating layer and a porous layer, where the porous layer is in contact with the first zone, such that the haematopoietic stem cells can be grown in the porous layer. The bioreactor comprises a third zone. The first zone and the third zone are separated by a second membrane, and the second membrane allows the passage of nutrients from the third zone to the first zone and the passage of metabolites of the cells from the first zone to the third zone, while substantially preventing the passage of growth factors from the first zone to the third zone.

Within the human body, a number of organs use a “hollow-fiber” type structure, through vascularisation, to accomplish their designated task. The main organs that could be replicated through this architecture are: bone marrow which regenerates blood cells and secretes important molecules; the liver which has a major role in metabolism and blood filtration; the pancreas which provides feedback control of glucose concentration in the blood; and the kidney which removes metabolic products from blood.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference, including the content of the priority application.

### REFERENCES

1. Cabral J; Ex vivo expansion of hematopoietic stem cells in bioreactors; Biotechnology Letters, 23 (10): 741-751, 2001.
2. Li Y et al.; Human Cord Cell Hematopoiesis in Three-Dimensional Nonwoven Fibrous Matrices: In Vitro Simulation of the Marrow Microenvironment; Journal of Hematotherapy & Stem Cell Research, 10 (3): 355-368, 2001.
3. Mantalaris A et al.; Engineering a human bone marrow model: A case study on ex vivo erythropoiesis; Biotechnol Prog, 14 (1): 126-133, 1998.
4. Pathi P et al.; Role of nutrient supply on cell growth in bioreactor design for tissue engineering of hematopoietic cells; Biotechnology and Bioengineering, 89 (7): 743-758, 2004.
5. Pathi P. Mathematical Modeling of Transport and Reaction in Cellular and Tissue Engineering. 2005.
6. Mantalaris A, Panoskaltsis N, Wu J. Tissue Engineering of Bone Marrow. Encyclopedia of Biomaterial and Biomedical Engineering. New York: Marcel Dekker, Inc., 2004: 1508-1517.
7. Ambrosio L, Netti P, Revell P. Tissues. Integrated Biomaterials Science. Springer U S, 2007: 339-345.
8. Hill A; The Diffusion of Oxygen and Lactic Acid through Tissues; Proceedings of the Royal Society of London, Series B, 104 (728): 39-96, 1928.
9. Ma C et al.; Development of a Mathematical Model for a 3-D Perfused Bone Marrow Culture System; Poster Session for AIChE: Biomaterials and Tissue Engineering, 2005.
10. Peng C A and Palsson B O; Determination of Specific Oxygen Uptake Rates in Human Hematopoietic Cultures and Implications for Bioreactor Design; Annals of biomedical engineering, 24: 373-381, 1996.
11. Chaudhuri J, Al-Rubeai M. Bioreactors for Tissue Engineering: Principles, Design and Operation. The Netherlands: Springer, 2005.
12. Knazek R et al.; Cell culture on artificial capillaries: An approach to tissue growth in vitro; Science, 178: 65-67, 2008.
13. Williams S et al.; Mapping of oxygen tension and cell distribution in a hollow-fiber bioreactor using magnetic resonance imaging; Biotechnol Bioeng, 56: 56-61, 1997.
14. Gramer M et al.; Effect of harvesting protocol on performance of a hollow fiber bioreactor; Biotechnol Bioeng, 65: 334-340, 1999.
15. Placzek M R, Chung I M, Macedo H M, Ismail S, Mortera B T, Lim M et al. Stem cell bioprocessing: fundamentals and principles. J R Soc Interface 2009; 6(32):209-232.
16. Cabrita G et al.; Hematopoietic stem cells: from the bone to the bioreactor; Trends Biotech, 21 (5): 233-240, 2003.
17. Koller M R et al.; Growth-Factor Consumption and Production in Perfusion Cultures of Human Bone-Marrow Correlate with Specific Cell Production; Experimental Hematology, 23 (12): 1275-1283, 1995.
18. Kim B; Production of human hematopoietic progenitors in a clinical-scale stirred suspension bioreactor; Biotechnol lett, 20 (6): 595-601, 1998.
19. Engelhardt M et al.; Hematopoietic recovery of ex vivo perfusion culture expanded bone marrow and unexpanded peripheral blood progenitors after myeloablative chemotherapy; Bone Marrow Transplantation, 27: 249-259, 2001.
20. Lee D E et al.; The prolonged half-lives of new erythropoietin derivatives via peptide addition; Biochemical and Biophysical Research Communications, 339 (1): 380-385, 2006.
21. Coles G A, Liberek T, Davies M E, Robinson M, Jones J, Thomas G et al. Estimation of erythropoietin secretion rate in normal and uremic subjects. Am J Physiol Renal Physiol 1992; 263(5):F939-F944.
22. Schmidt N M, Schmidt R A, Teramo K A, Widness J A. Endogenous and Recombinant Erythropoietin Levels Decline in Human Amniotic Fluid and Fetal Plasma in vitro at 37[deg]C. J Perinatol 2004; 24(4):218-222.
23. Kaushansky K. Lineage-Specific Hematopoietic Growth Factors. The New England Journal of Medicine 2006; 354(19):2034-2045.
24. Zandstra P et al.; Cellular determinants affecting the rate of cytokine in cultures of human hematopoietic cells; Biotechnol Bioeng, 54 (1): 58-66, 1997.
25. Uppenkamp M. Thrombopoietin serum concentration in patients with reactive and myeloproliferative thrombocytosis. Annals of hematology 1998; 77(5):217-223.
26. Langley K E, Bennett L G, Wypych J, Yancik S A, Liu X D, Westcott K R et al. Soluble stem cell factor in human serum. Blood 1993; 81(3):656-660.
27. Kaushansky K; Thrombopoietin; N Engl J Med, 339 (11): 746-754, 1998.
28. Curcio E et al.; Diffusive and convective transport through hollow fiber membranes for liver cell culture; Journal of Biotechnology, 117: 309-321, 2005.

1. A bioreactor for the formation of mature blood cells from haematopoietic stem cells, the bioreactor comprising a first zone and a second zone, wherein the first zone and the second zone are separated by a first membrane, wherein the first membrane allows the preferential passage of red blood cells relative to the haematopoietic stem cells and their other progeny excluding red blood cells, wherein the first membrane is formed by at least a separating layer and a porous layer, where the porous layer is in contact with the first zone, such that the haematopoietic stem cells can be grown in the porous layer.

2. The bioreactor as claimed in claim 1, wherein the bioreactor comprises a third zone, wherein the first zone and the third zone are separated by a second membrane, and the second membrane allows the passage of nutrients from the third zone to the first zone and the passage of metabolites of the cells from the first zone to the third zone, while substantially preventing the passage of growth factors from the first zone to the third zone.

3. The bioreactor as claimed in claim 1, wherein the porous layer has a three-dimensional shape and the first membrane and/or the second membrane is located within the three-dimensional shape.

4. The bioreactor as claimed in claim 1, wherein the first membrane and/or the second membrane is in the form of a hollow fibre.

5. A bioreactor comprising a first zone defined by a porous layer having a three-dimensional shape, a second zone defined by a first membrane in the form of a hollow fibre located within the porous layer, and a third zone defined by a second membrane in the form of a hollow fibre located within the porous layer, whereby the first zone and the second zone are separated by the first membrane and the first zone and the third zone are separated by the second membrane, and wherein the porosity of the first membrane is greater than the porosity of the second membrane, such that the second membrane will retain cells or molecules within the first zone that will pass through the first membrane.

6. (canceled)

7. The bioreactor as claimed in claim 1, wherein the porous layer is a polymeric scaffold composed of at least one of the following polymers: polyurethane, poly (L-lactic-co-glycolic acid), poly (methylmethacrylate), poly (D, L-lactate), poly (caprolactone), polystyrene and derivatives thereof.

8. (canceled)

9. The bioreactor as claimed in claim 1, wherein said first membrane or said second membrane is inorganic, preferably being formed of alumina oxide, titania oxide, zirconia oxide, glassy materials, or derivatives thereof.

10. The bioreactor as claimed in claim 1, wherein said first membrane or said second membrane is organic, preferably being formed of polyacrylonitrile, polyimide, polyamide, polyurethane, poly (L-lactic-co-glycolic acid), poly (methylmethacrylate), poly (D, L-lactate), poly (caprolactone), polystyrene, polyether ether ketone, polyethersulphone, polyvinylidene fluoride, or derivatives thereof.

11. The bioreactor as claimed in claim 9, wherein said first membrane is inorganic and said second membrane is organic.

12. (canceled)

13. (canceled)

14. (canceled)

15. (canceled)

16. (canceled)

17. (canceled)

18. The bioreactor as claimed in claim 1, wherein said second membrane has a molecular weight cut-off ranging between 1,000 and 30,000 Da, more preferably between 2,000 and 25,000 and even more preferably between 3,000 and 20,000.

19. (canceled)

20. (canceled)

21. (canceled)

22. (canceled)

23. (canceled)

24. (canceled)

25. (canceled)

26. (canceled)

27. (canceled)

28. A process for the formation of mature blood cells incorporating a bioreactor as claimed in claim 1, wherein the first zone contains a mixture of cells including haematopoietic stem cells and their progeny including red blood cells, wherein the first membrane allows the preferential passage of red blood cells relative to the haematopoietic stem cells and their other progeny excluding red blood cells, wherein the porous layer is in contact with the first zone, such that the haematopoietic stem cells grow in the porous layer.

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. A bioreactor as claimed in claim 1, wherein said first membrane, said second membrane and/or said porous layer is modified in order to attach at least one of peptides, growth factors, cytokines that are known to promote angiogenesis, cytokines such as vascular endothelial growth factor (VEGF) or modified to inhibit cell growth or to incorporate bioactive signals for the culture of endothelial cells.

49. The process for the formation of mature blood cells as claimed in claim 1, wherein the bioreactor has a third zone, wherein the second membrane allows the passage of nutrients from the third zone to the first zone and the passage of metabolites of the cells from the first zone to the third zone, while substantially preventing the passage of growth factors from the first zone to the third zone.

50. The process as claimed in claim 49, wherein one of a cytokine-free environment, a serum-free environment, one or more cytokines or human or animal-derived serum is/are used.

51. The process as claimed in claim 12, wherein said first membrane is used to provide cytokines, nutrients, oxygen and any other important molecules for the metabolic and functional activity of the cells being grown, or wherein said first membrane is used to selectively harvest mature or nearly-mature blood cells from said first zone into said second zone, which optionally are afterwards collected in a sterile container.

52. A process as claimed in claim 49, wherein said second membrane is used to provide small molecules, such as nutrients to the first zone and to remove small molecules such as cellular metabolites from the first zone.

53. A process as claimed in claim 12, wherein angiogenesis is promoted within said porous layer in order to further enhance the transport properties of the same.

54. The use of the bioreactor as claimed in claim 1 for:
the culture of human haematopoietic stem cells with a view to the expansion of stem cells as well as the production of progenitors, precursors and mature haematopoietic stem cells; the culture of human stem cells for the expansion of stem cells as well as the production of progenitors, precursors and mature cells derived from them; culturing haematopoietic stem cells using growth factors in the third zone, wherein the flow rate of the second zone is higher than the flow rate of the third zone by a factor of 1 to 100; culturing stem cells using growth factors by recycling the growth factors in the third zone and renewing the second zone; the production of human red blood cells from a source of stem cells, such as umbilical cord blood stem cells, induced pluripotent stem cells, embryonic stem cells, bone marrow stem cells and peripheral blood stem cells; or culturing stem cells using an oxygen concentration in the first zone ranging from 1% to 21%; or, for:
the production of human platelets from a source of stem cells, such as umbilical cord blood stem cells, induced pluripotent stem cells, embryonic stem cells, bone marrow stem cells and peripheral blood stem cells; the production of human white blood cells from a source of stem cells, such as but umbilical cord blood stem cells, induced pluripotent stem cells, embryonic stem cells, bone marrow stem cells and peripheral blood stem cells; or
the culture of human leukemic stem cells from either immortalized cell lines or harvested from human patients.
